(19) United States
(12) Patent Application Publication
(10) Pub. No.: US 2020/0352136 A1
(43) Pub. Date: Nov. 12, 2020

(54) METHODS AND SYSTEM FOR PET ENRICHMENT

(71) Applicants: Kevin Hanson, San Tan Valley, AZ (US); Victoria Ramos, Chandler, AZ (US); Kyle Alexander, Boise, ID (US); Hettie Haines, Phoenix, AZ (US)

(72) Inventors: Kevin Hanson, San Tan Valley, AZ (US); Victoria Ramos, Chandler, AZ (US); Kyle Alexander, Boise, ID (US); Hettie Haines, Phoenix, AZ (US)

(21) Appl. No.: 16/939,963

(22) Filed: Jul. 27, 2020

Related U.S. Application Data
(63) Continuation-in-part of application No. 15/143,507, filed on Apr. 30, 2016, now Pat. No. 10,721,912.

(51) Int. Cl. A01K 15/02; G06Q 50/00; G16H 50/20; G06N 20/00; G06T 7/521; G06T 7/73; G06T 7/246; G06T 7/90; A01K 29/00; A61B 5/00; A61B 5/0205; A61B 5/11

(52) U.S. Cl. CPC A01K 15/021; G06Q 50/01; G16H 50/20; G06N 20/00; G06T 7/521; G06T 7/73; G06K 9/00335; G06T 7/90; A01K 29/005; A61B 5/0022; A61B 5/02055; A61B 5/11; G06T 7/246

(57) ABSTRACT

A system for interacting with an animal provides a local device to provide a cue to the animal and generate characteristic data of the animal, a remote network in communication with the local device and configured to receive the characteristic data and generate first feedback data, and execute a machine learning algorithm to generate learned data based on the first feedback data and the characteristic data; wherein the local device is further configured to operate according to the learned data.

### CROSS REFERENCE TO RELATED APPLICATIONS

This application is a continuation-in-part of U.S. patent Ser. No. 15/143,507 filed on Apr. 30, 2016, which claims the benefit of U.S. Provisional Patent Application Ser. No. 62/155,439, filed on Apr. 30, 2015, and incorporates the disclosure of the application in its entirety by reference. To the extent that the present disclosure conflicts with any referenced application, however, the present disclosure is to be given priority.

### BACKGROUND

Embodiments disclosed in the present technology relate generally to pet training, and more specifically to an automatic pet training device and method of operating same.

Conventional pet training is performed in-person by the pet owner or professional pet trainer. Training can be done one-on-one with individual animals or with may be performed in a group setting. Pet training, especially for younger animals, requires many hours, spent daily, to train not only the pet's behavior, but also to train the pet to do tricks, such as sit, lay, turn around, etc. Proper training requires constant positive reinforcement, which is usually though the use of edible objects or treats. Training at this early stage can be quite time consuming for the owners, especially if the owner works outside the home. Pets that do not have constant reinforcement of what they are learning may not learn as quickly.

Additionally, some pets exhibit behavioral problems when left alone. Destructive or aggressive behavior may be the result of boredom and/or separation anxiety when the pet is left alone. Owners have limited choices to address these issues. One option is to take their pet to a boarding facility during the day. However, this option can be rather costly. Other options, such as asking friends or family to watch the animal all day, may not be feasible for every pet owner.

Accordingly, it would be desirable to have an affordable pet training device that reinforces a particular behavior, such as performing tricks, as well as provides the animal with an activity to reduce boredom and alleviate separation anxiety.

### BRIEF DESCRIPTION OF THE DRAWINGS

FIG. 1 representatively illustrates a pet enrichment device in accordance with an embodiment of the present technology;

FIG. 2 representatively illustrates a pet enrichment device in accordance with an embodiment of the present technology;

FIG. 3 illustrates a flowchart in accordance with an embodiment of the present technology;

FIGS. 4A-4C representatively illustrate various detectable positions and movements in accordance with an embodiment of the present technology;

FIGS. 5A-5B representatively illustrate an animal with wearable color blocking in accordance with an embodiment of the present technology;

FIG. 6 representatively illustrates a cross section of a treat dispenser in accordance with an embodiment of the present technology;

FIG. 7 representatively illustrates a system in accordance with an embodiment of the present technology;

FIG. 8 representatively illustrates machine learning in accordance with an embodiment of the present technology; and

FIG. 9-12 illustrate a flow chart for operating the system of FIG. 7 in accordance with an embodiment of the present technology.

For simplicity and clarity of illustration, elements in the figures are not necessarily drawn to scale, and the same reference numbers in different figures denote generally the same elements.

### DETAILED DESCRIPTION OF THE DRAWINGS

The present technology may be described in terms of functional block components and various processing steps. Such functional blocks may be realized by any number of components configured to perform the specified functions and achieve the various results. For example, the present technology may employ various processors, controllers, timing devices, detection units, semiconductor devices, switching devices, and the like, which may carry out a variety of functions. In addition, the present technology may be practiced in conjunction with any animal training and/or activity system, and the device and method described are merely exemplary applications for the technology.

Methods and device for pet enrichment according to various aspects of the present technology may operate in conjunction with a training tool, such as teaching the animal to perform particular commands, for example, sit, lay down, spin, etc. Methods and device for pet enrichment according to various aspects of the present technology may operate in conjunction with a positive reinforcement system, for example, providing a reward, such as an edible object (treat). Methods and device for pet enrichment according to various aspects of the present technology may operate in conjunction with an entertainment system, for example providing interactive activities such as fetching a particular object. Methods and device for pet enrichment according to various aspects of the present technology may provide positive reinforcement to the animal.

The device 100 may comprise various components configured to perform target recognition, analysis, and tracking. In various embodiments, the device 100 may detect the presence of an animal. For example, in one embodiment, the device 100 may detect that an animal is within a range of distance from the device 100. In various embodiments, the device 100 may detect and identify specific movements and/or activity of the animal. For example, the device 100 may detect and identify if the animal is spinning around or jumping. In various embodiments, the device 100 may detect the specific position (posture) of the animal, for example if the animal is sitting, standing, or laying down.

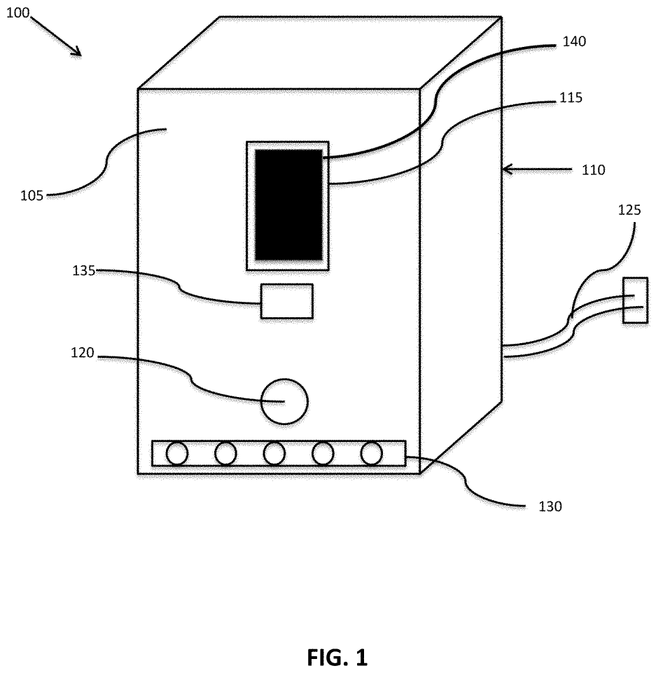

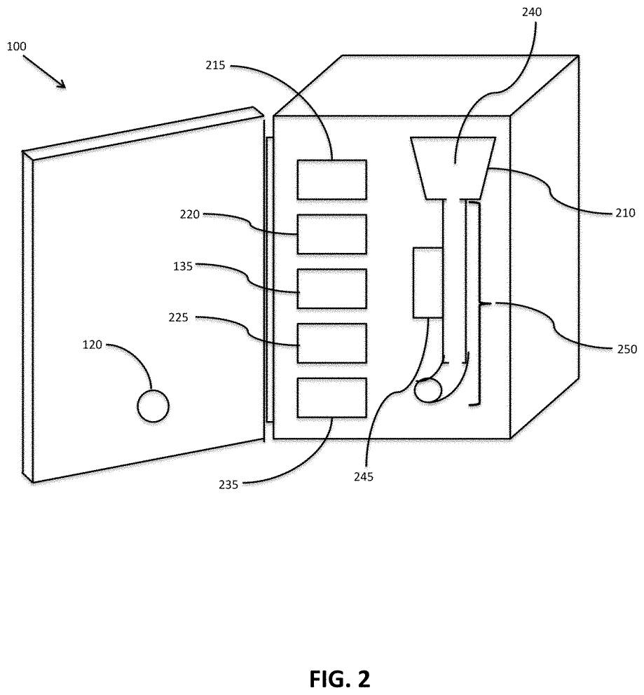

Referring to FIG. 1, in various embodiments, methods and device 100 for pet enrichment may comprise a housing 105. In various embodiments, the housing 105 may comprise one or more surfaces forming an exterior portion 110, wherein at least one surface is movable, for example by a hinge, or removable by screws or any other suitable fastener. For example, the housing 105 may comprise eight (8) surfaces formed in the shape of a cube or rectangle. In various embodiments, the one of more surfaces may also form an interior portion 200 (FIG. 2). In various embodiments, the interior portion may be accessed by the movable surface.

In various embodiments the housing 105 may be constructed from a solid material, such as wood, plastic, metal, particle board, and/or any other type of durable materials. In various embodiments, the housing 105 may be constructed of two or more types of solid durable materials for added structural integrity. In various embodiments, the housing 105 may be constructed to stand upright while sitting on a floor, or may be affixed to a vertical surface, such as a wall, or door.

In various embodiments, the device 100 may comprise one or more openings 120 in the housing 105. In various embodiments, as least one opening 120 may provide an exit for edible objects. In various embodiments, the opening 120 is positioned near the bottom of the housing 105, but may be positioned in any suitable location.

In various embodiments, the device 100 may comprise a power supply. In one embodiment, the power supply may comprise an electrical connection 125 to an external power supply, such as a regular electrical outlet. In other embodiments, the power supply may comprise one or more batteries (not shown), for example, rechargeable lithium batteries.

The device 100 may comprise an interface device 115. In various embodiments, the interface device 115 may comprise a screen 140 for displaying video content, and a speaker (not shown) for projecting audio content. In various embodiments, the interface device 115 may comprise an electronic device, such as a tablet, iPad, or the like. In various embodiments, the interface device 115 may produce a cue, via the screen 140 and/or speaker, for the animal, where the cue provides the animal with a command. For example, the interface device 115 may display a video using hand gestures (i.e. sign language) to instruct the animal to perform some command, such as come here, sit, lay down, spin, speak (bark), or any other command that a user wishes the animal to perform. The interface device 115 may also emit an audio cue, via the speaker, to instruct the animal to perform a command such as come here, sit, lay down, spin, speak (bark), or any other command that a user wishes the animal to perform. In various embodiments, the audio cue may comprise voice commands and/or “clicker” noises. In various embodiments, cues from both video and audio sources are produced simultaneously. In various embodiments, the interface device 115 comprises a camera (not shown) communicatively coupled to the screen 140 and/or speaker. In various embodiments, the interface device 115 comprises a communication unit (not shown), for example a wireless communication unit such as Bluetooth, to communicate with the device 100.

In an exemplary embodiment, the video and audio cues may be pre-recorded by an owner and/or a handler of the animal. For example, the owner may record him or herself giving a cue in the form of audio of visual cues. In one embodiment, the video and audio cues may be streamed to the interface device 115 via a network. In other embodiments, the video and audio cues may be saved directly to the interface device 115 and accessible by the device 100. In various embodiments, the cues are generated at predetermined times throughout the day, or may be generated at random times.

In various embodiments, the interface device 115 may provide positive reinforcement to the animal. For example, in various embodiments, the interface device 115 may project an image on the screen 140 in conjunction with an audio component, wherein the audio projects a complimentary message, such as “good dog.”

In various embodiments, the device 100 may comprise a lighting source 130. In various embodiments, the lighting source 130 may be electrically connected to a relay mechanism (not shown) to provide dimming capability for the light source 130. In various embodiments, the light source 130 may be activated based on the external lighting conditions. For example, the light source 130 may be activated if the lighting levels are below a predetermined threshold value. In other embodiments, the light source 130 may be activated based on the activation of one or more other components of the device 100.

In an exemplary embodiment, the lighting source 130 may be affixed to the front of the housing body. The lighting source 130 may provide illumination to a predetermined area in front of the device 100. In one embodiment, the light source 130 may be affixed to the front lower half of the housing 105, however may be located in any suitable location to provide illumination to a desired area. The lighting source 130 may comprise an LED (light emitting diode) strip with multiple LEDs, or may comprise any other suitable light. The light source 130 may be selectively illuminated when the ambient light is below a predetermined level. The light source 130 may be activated at substantially the same time as various other components of the device 100. For example, the light source 130 may be activated at substantially the same time as the interface device 115 if the device 100 detects low lighting conditions of the ambient light.

In various embodiments, the device 100 may further comprise a clicker device to generate a “clicker” sound for providing various commands. In other embodiments, the interface device 115 may generate the “clicker” sound for the same or similar purpose.

In various embodiments, the device 100 may also be communicatively coupled to a network, for example the internet, so that the device 100 may be accessed remotely to provide cues to the animal. For example, the owner may be able to log into the device 100 remotely to activate the device 100 and provide any one of the pre-recorded cues to the animal and/or positive reinforcement.

In various embodiments, the device 100 may comprise an application and equipment for transmitting and receiving video. For example, the device 100 may operate in conjunction with Skype or a Skype-like application and a webcam.

Referring to FIG. 2, in various embodiments, the device 100 may comprise a treat dispenser 210 to provide positive reinforcement.

In various embodiments, the treat dispenser 210 may comprise a chamber 240, a channel 250, and a motorized mechanism 245. The treat dispenser 210 may be configured to hold multiple edible objects and may be configured to dispense one or more edible objects at a time. The treat dispenser 210 may be constructed of plastic, metal, glass, or any other suitable food-grade material.

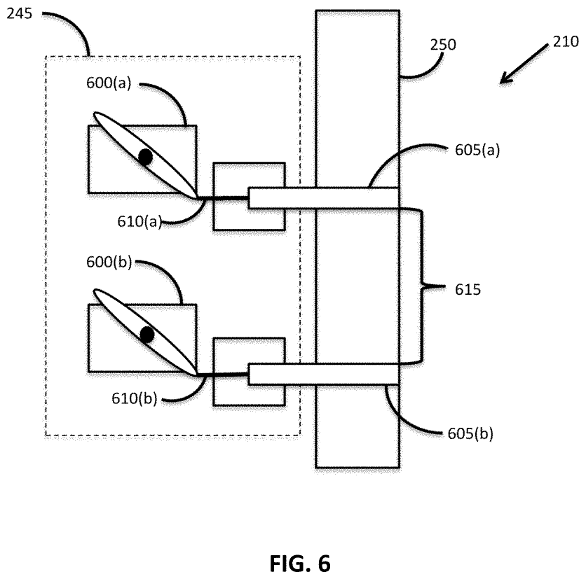

Referring to FIG. 6, in an exemplary embodiment, the motorized mechanism 245 may comprise at least two (2) micro motors 600(a), 600(b), two (2) vertically slidable components 605(a), 605(b), two (2) fasteners 610(a), 610(b), and two (2) springs (not shown). In one embodiment, each vertically slidable component 605(a), 605(b) is coupled to one of the micro motors 600 via one of the fasteners 610. In various embodiments, each spring may be movably coupled to an end of the vertically slidable components 605(a), 605(b) opposite that of the micro motors 600(a), 600(b) and fasteners 610(a), 610(b). The micro motors 600(a), 600(b) may be communicatively coupled to receive timing signals.

During operation, the micro motors 600(a), 600(b) may be timed such that a first slidable component 605(a) slides to the left (open), via a first micro motor 600(a) and a first fastener 610(a), to allow a treat to fall into an intermediate area 615, and then it slides back to the right (closed), via a first micro motor 600(a) and the first fastener 610(a) and/or in conjunction with a spring. Then, a second slidable component 605(b) slides to the left (open), via a second micro motor 600(b) and a second fastener 610(b), to allow a treat to fall out of the intermediate area 615, where the channel 250 directs the treat out of the opening 120 (FIG. 1). The second slidable component 605(b) then it slides back to the right (closed), via the second micro motor 600(b) and the second fastener 610(b) and/or in conjunction with a spring.

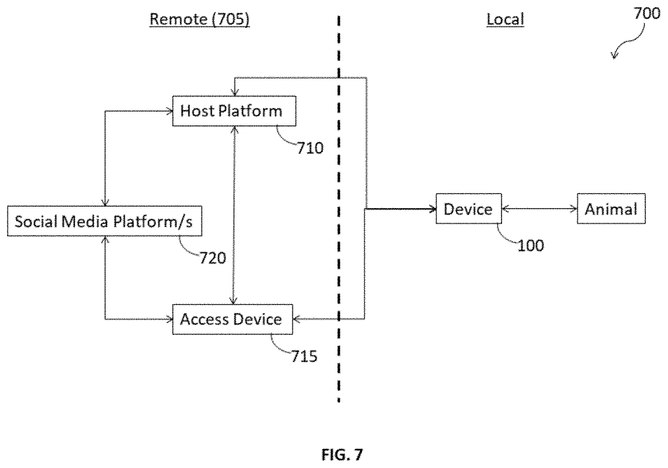

Referring back to FIG. 2, in various embodiments, the device 100 may further comprise a wireless communicator 220. In an exemplary embodiment, the device 100 may comprise one or more wireless communicators 220. The wireless communicator 220 may send and receive wireless signals. In one embodiment, the wireless communicator 220 communicates, via the wireless signals, with the interface device 115 (FIG. 1). The wireless communicator 220 may also communicate, via wireless signals, with a remote access device 715 (FIG. 7). For example, the wireless signals may be the form of infrared light or radio waves. In various embodiments, the remote access device 715 may comprise a hand-held device configured to send radio waves, such as a cellphone, laptop, computing pad, or any other device configured to communicate via wireless signal.

In various embodiments, the device 100 may further comprise a microcontroller 215. The microcontroller 215 may comprise a single integrated circuit containing a processor core, memory, and programmable input/output peripherals. In other embodiments, the processor and memory may be formed on separate chips. The microcontroller 215 may be programmed to initialize the interface device 115 (FIG. 1), store data received from the data acquisition unit 135, and operate the treat dispenser 210. The microcontroller 215 may comprise various hardware devices, semiconductor components, and/or wiring schemes to perform logic operations suitable to a particular embodiment. In various embodiments, the microcontroller 215 may be electrically connected to the light source 130 and may control illumination of the light source 130. In one embodiment, the microcontroller 215 may be programmed to initialize the interface device 115 via a wireless communicator 220. In other embodiments, the microcontroller 215 may initialize the data acquisition unit 135, and the treat dispenser 210, via an electrical connection. In various embodiments, the microcontroller 215 may be electrically connected to the power distribution device 220.

The microcontroller 215 may comprise a central processing unit and memory. The microcontroller 215 may receive and process communication signals from other components of the device 100. The microcontroller 215 may be formed on a single integrated circuit and may comprise a central processing unit, local memory, and programmable input/output peripherals. For example, the microcontroller 215 may provide ROM, EPROM, EEPROM, or flash memory. The microcontroller 215 may be configured to send and receive data from other components of the device 100 via input/output peripherals. The input/output peripherals may provide an electrical connection providing power and data to the components connected to the microcontroller 215. For example, the microcontroller 215 may be programmed using any suitable programming language, for example, C, C++, Java, Python, PHP, JavaScript, Ruby, and SQL. In some embodiments, the microcontroller 215 may be individually addressable. In some embodiments, the microcontroller 215 may be equipped with a UART or a USART to transmit and receive communication to and from a peripheral device port.

In various embodiments, the device 100 may further comprise a microphone 235. The microphone 235 may be configured to detect a sound above a certain frequency or decibel level. For example, in one embodiment, the microphone 235 is configured to detect the bark of an animal. The microphone 235 may be positioned in any location within the device 100. The microphone 235 may be coupled to the microcontroller 215.

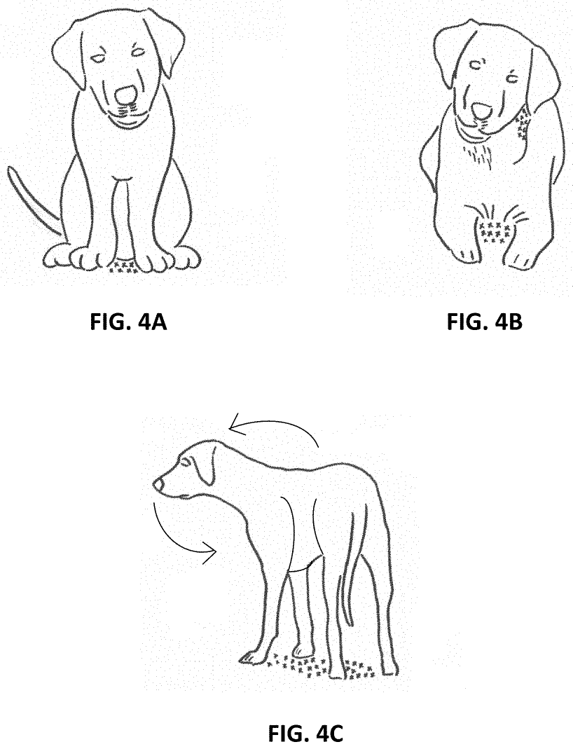

In various embodiments, the device 100 may comprise a data acquisition unit 135. In various embodiments, the data acquisition unit 135 may comprise any number of components to recognize, analyze, and track the animal to determine the specific position (posture) and/or movements of the animal, for example, a sit position, as illustrated in FIG. 4A, a laying position, as illustrated in FIG. 4B, and a spinning motion, as illustrated in FIG. 4C. For example, the data acquisition unit 135 may comprise one integrated device to perform recognition, analysis, and tracking, while in other embodiments, the data acquisition unit may comprise multiple devices to perform recognition, analysis, and tracking, each device communicatively coupled and performing separate functions. The data acquisition unit 135 may be partially or substantially integrated in the housing 105.

In various embodiments, the data acquisition unit 135 may comprise various 3D mapping technology and methods, for example, the 3D mapping methods as described in U.S. Application No. 2010/0199228, U.S. Application No. 2010/0201811, U.S. Application No. 2010/0195867, U.S. Application No. 2010/0118123, U.S. Application No. 2013/0038941, which are incorporated by reference herein.

In various embodiments, the data acquisition unit 135 may comprise a lens or lenses, and may be integrated in the housing 105 such that the lens or lenses protrude through the front of the housing 105 while a substantial portion of the data acquisition unit 135 may be located within the interior of the device 100.

In one embodiment, the data acquisition unit 135 may comprise a color-detecting camera. For example, the camera may be programmed to detect predefined colors. These predefined colors may be worn by the animal and configured for optimum detection. For example, the animal may wear a harness 25 having a color blocking configuration with multiple colors. Other wearable color blocks may be implemented, such as colored booties, color blocked vests, color blocked shirts, or color blocked collars. In various embodiments, the camera may determine x-y-z coordinates of each of the color blocks. The x-y-z coordinates may be tracked and stored over a period of time, for example 10 seconds. The microcontroller 215 may analyze the x-y-z coordinates to determine the specific position of the animal based on the x-y-z coordinates of the color blocks, and may determine the specific movements of the animal based on the x-y-z coordinates of the color blocks over the specified period of time.

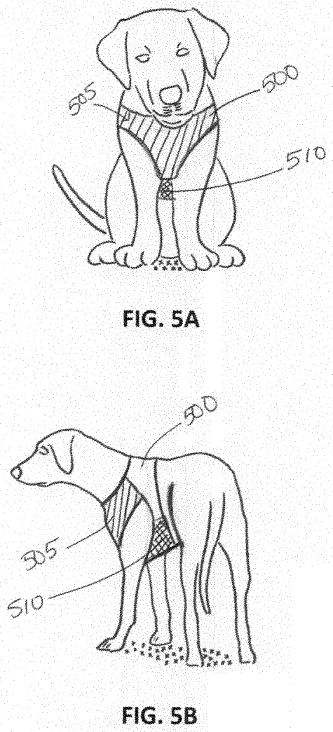

Referring to FIGS. 5A-5B illustrates an animal with wearable color blocking in accordance with one embodiment. The figures illustrate the use of color blocking on a harness 500. As illustrated, the harness 500 may have two blocks of color. The harness 500 may comprise a pink color block 505, and a green color block 510. In one embodiment, where a color-detection camera is used for the data acquisition unit 135, the color-detection camera may be programmed to detect only the pink and green colors of the harness 500. Other configurations using color blocks, such as orange, blue, purple, or any other color may be implemented, and configurations using more than two colors may also be implemented.

The data acquisition unit 135 may comprise a depth sensor, for example a depth camera, using an infrared (IR) projector and a monochrome CMOS (complementary metal oxide semiconductor) sensor. For example, the infrared projector may project a pattern of infrared light and the distortion, which is caused when the pattern becomes distorted by light, is read by the depth camera. The depth camera may then analyze the IR patterns to build a 3-D map of the room and all objects within it.

The device 100 may comprise a computing environment. The computing environment may comprise hardware components and/or software components such that the computing environment may be used to execute applications, such the interface device 115. The computing environment may comprise a central processing unit (CPU) having a level 1 cache, a level 2 cache, and a flash ROM (Read Only Memory). The level 1 cache and a level 2 cache temporarily store data and hence reduce the number of memory access cycles, thereby improving processing speed and throughput. The CPU may be provided having more than one core, and thus, additional level 1 and level 2 caches. The flash ROM may store executable code that is loaded during an initial phase of a boot process when the multimedia console is powered ON.

The data acquisition unit 135 may comprise may a camera that may be used to visually monitor the target, such as the animal, such that specific positions and/or movements performed by the animal may be captured, analyzed, and tracked to activate the treat dispenser 210.

The animal may have to retrieve an object ejected from the device 100. In such embodiments, the animal may be holding the object such that the motions of the animal and the object may be used to adjust and/or control parameters the interface device 115. For example, the animal may be holding a ball in his or her mouth and the interface device 115 may instruct the animal to “drop” the ball.

According to an exemplary embodiment, the data acquisition unit 135 may be configured to capture video with depth information including a depth image that may include depth values via any suitable technique including, for example, time-of-flight, structured light, stereo image, or the like. According to one embodiment, the data acquisition unit 135 may organize the calculated depth information into “Z layers,” or layers that may be perpendicular to a Z axis extending from the depth sensor along its line of sight.

The data acquisition unit 135 may comprise an image camera component. According to an example embodiment, the image camera component may be a depth camera that may capture the depth image of a scene. The depth image may include a two-dimensional (2-D) pixel area of the captured scene where each pixel in the 2-D pixel area may represent a length in, for example, centimeters, millimeters, or the like of an object in the captured scene from the camera.

According to an example embodiment, the image camera component may comprise an IR light component, a three-dimensional (3-D) camera, and an RGB camera that may be used to capture the depth image of a scene. For example, in time-of-flight analysis, the ER light component of the data acquisition unit 135 may emit an infrared light onto the scene and may then use sensors (not shown) to detect the backscattered light from the surface of one or more targets and objects in the scene using, for example, the 3-D camera and/or the RGB camera. In some embodiments, pulsed infrared light may he used such that the time between an outgoing light pulse and a corresponding incoming light pulse may be measured and used to determine a physical distance from the data acquisition unit 135 to a particular location on the targets or objects in the scene. Additionally, in other example embodiments, the phase of the outgoing light wave may be compared to the phase of the incoming light wave to determine a phase shift. The phase shift may then be used to determine a physical distance from the data acquisition unit 135 to a particular location on the targets or objects.

According to another example embodiment, time-of-flight analysis may be used to indirectly determine a physical distance from the data acquisition unit 135 to a particular location on the targets or objects by analyzing the intensity of the reflected beam of light over time via various techniques including, for example, shuttered light pulse imaging.

In another example embodiment, the data acquisition unit 135 may use a structured light to capture depth information. In such an analysis, patterned light (i.e., light displayed as a known pattern such as grid pattern or a stripe pattern) may be projected onto the scene via, for example, the IR light component. Upon striking the surface of one or more targets or objects in the scene, the pattern may become deformed in response. Such a deformation of the pattern may be captured by, for example, the 3-D camera and/or the RGB camera and may then be analyzed to determine a physical distance from the data acquisition unit 135 to a particular location on the targets or objects.

According to another embodiment, the data acquisition unit 135 may comprise two or more physically separated cameras that may view a scene from different angles, to obtain visual stereo data that may be resolved to generate depth information.

The data acquisition unit 135 may further comprise a processor that may be in communication with the image camera component. The processor may comprise a standardized processor, a specialized processor, a microprocessor, or the like that may execute instructions that may include instructions for receiving the depth image, determining whether a suitable target may be included in the depth image, converting the suitable target into a skeletal representation or model of the target, or any other suitable instruction.

The device 100 may further comprise a memory component that may store the instructions that may be executed by the processor, images or frames of images captured by the 3-D camera or RGB camera, or any other suitable information, images, or the like. According to an example embodiment, the memory component may include random access memory (RAM), read only memory (ROM), cache, Flash memory, a hard disk, or any other suitable storage component. In one embodiment, the memory component may be a separate component in communication with the image capture component and the processor. According to another embodiment, the memory component may be integrated into the processor and/or the image capture component.

Additionally, the data acquisition unit 135 may provide the depth information and images captured by, for example, the 3-D camera and/or the RGB camera, and a skeletal model of the animal that may be generated by the data acquisition unit 135 to the computing environment via the communication link. The computing environment may then use the skeletal model, depth information, and captured images to recognize the animal's position and movement (i.e. gestures) in response to cues generated by the interface device 115. For example, the computing environment may include a gestures recognizer engine. The gestures recognizer engine may include a collection of gesture filters, each comprising information concerning a gesture that may be performed by the skeletal model (as the animal moves). The data captured by the cameras and device in the form of the skeletal model and movements associated with it may be compared to the gesture filters in the gesture recognizer engine to identify when the animal (as represented by the skeletal model) has performed one or more gestures. Those gestures may be associated with various controls of an application. Thus, the computing environment may use the gesture recognizer engine to interpret movements of the skeletal model and to control an application based on the movements.

A graphics processing unit (GPU) and a video encoder/video codec (coder/decoder) may form a video processing pipeline for high speed and high resolution graphics processing. Data is carried from the graphics processing unit to the video encoder/video codec via a bus. The video processing pipeline may output data to an A/V (audio/video) port for transmission to the interface device 115 or other display. A memory controller may be connected to the GPU to facilitate processor access to various types of memory, such as, but not limited to, a RAM (Random Access Memory).

In an alternative embodiment, the data acquisition unit 135 may comprise a pressure sensor, for example a pressure sensitive pad. In various embodiments, the pressure sensitive pad may comprise sensors to detect pressure, such as capacitive sensing and weight sensing. In various embodiments, the pressure sensitive pad may be coupled to the microcontroller 215. The pressure sensitive pad may transmit signals generated by the sensors to the microcontroller 215 and/or computing environment, wherein the location of the generated signals may be analyzed to determine the specific position and/or specific movement of the animal.

In another embodiment, the data acquisition unit 135 may provide wireless transmission/reception of data from one or more sensors, such as a gyroscope, a compass, an accelerometer, a radio-frequency identification (“RFID) chip, barometric pressures, and the like. Such sensors may be integrated into a wearable, such as a collar or a harness and signals from the sensors may be analyzed to determine the specific position (i.e., posture) and/or specific movement of the animal. The wearable device may comprise a power source, such as a battery.

In various embodiments, the animal may be provided a wearable detection unit equipped with various biosensors to monitor and/or measure biometrics of the animal such as temperature, heart rate, blood pressure, sleep patterns and the like. The biosensors may be integrated into wearable, such as a collar, a harness, a strap, or any other suitable mechanism. The wearable detection unit may be configured to transmit signals from the biosensor to the microcontroller 215 and/or computing environment, wherein signals may be analyzed to determine the health of the animal. The wearable device may comprise a power source, such as a battery.

Wearable devices may also provide a mechanism for distinguishing between multiple animals.

The device 100 may further comprise a power distribution device 225 to distribute power to various components, such as the microcontroller 215, the interface device 115 (FIG. 1), the data acquisition unit 135, and the like. The power distribution device 225 may comprise multiple outlets, such as USB outlets, and any other outlets for providing an electrical connection.

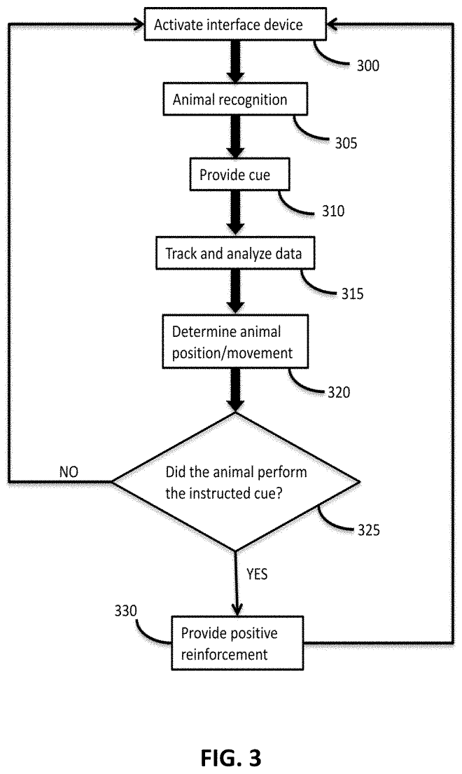

Referring to FIG. 3, in operation, the device 100 may activate 300 via the microcontroller 215 or other processor. Activation 300 may comprise initializing the interface device 115, wherein activation 300 may be performed at set times or activation 300 may be performed at random times. In various embodiments, the data acquisition unit 135 may then perform recognition 305 to determine whether or not the animal is present. If the data acquisition unit 135 determines that the animal is present 305, then the interface device 115 may provide a cue 310, such as displaying a video and/or audio cue, to instruct the animal to perform some command, such as sit or lay down. After the cue is presented to the animal, the device 100 may then analyze data from the data acquisition unit 135 and track 315 the animal to determine the specific position (posture) and/or movements of the animal. If the animal performs the instructed cue 325, the animal may receive some form of positive reinforcement 330, such as a treat via the treat dispenser 310 or complimentary message via the interface device 115 and/or speaker. If the animal does not perform the instructed cue 325, then the device 100 may begin a new activation 300 as described above.

In various embodiments, the device 100 (FIG. 1) may record and log the animal's performance. For example, the device 100 may record the number of times a cue as provided and the number of times the animal successfully performed the given cue. In various embodiments, the device 100 may also record and track the performance of particular cues for progressive training purposes. For example, the animal may be given one or two basic cues when the animal is first introduced to the device 100 and then increase the level of difficulty as the animal shows increased performance. The owner may then be able to access the logged data, by downloading the data or any other suitable method to view the animal's progress. In various embodiments, the animal may be recorded via a camera operable as a video camera for recording the animal as it performs the cues. For example, the recorded video may be viewed at a later time by the owner or to update and modify the device 100.

In various embodiments, operation of the device 100 may be controlled remotely by the owner. The owner may select and provide a specific cue, for example one of the pre-recorded cues, via the interface device 115, to the animal. The owner may receive a live streaming video of the animal while it performs the cue to access the animal's performance. In various embodiments, the owner may be able to provide positive reinforcement remotely. Positive reinforcement may be provided if the animal performs the given cue correctly, or for no reason at all.

A system 700 for interacting with the animal may comprise the device 100 and a number of remotely-connected devices and/or platforms. For example, the system 700 may comprise a remote network 705 that is in communication with (e.g., wirelessly connected to) the device 100. In various embodiments, the device 100 interacts with the remote network 705 to allow remote operation of the device 100, to remotely process data from the device 100, exchange data with the device 100, enhance various functions/operations of the device 100, and provide an environment for sharing various data and facilitating feedback data.

In various embodiments, the remote network 705 may comprise a host processor 710 (i.e., host platform) configured to store data, facilitate data transmission, perform various processing functions, and the like. The host processor 710 may be realized through cloud computing, hardware, software, or any combination thereof. For example, the host processor 710 may receive various characteristic data from the device 100, such as data from the image sensor and/or other sensors contained within the device 100. The characteristic data may comprise image data of the animal, audio data of the animal, orientation data of the animal (i.e., location of the animal relative to the device 100 or absolute location via GPS), position data of the animal (i.e., body position/posture of the animal), acceleration data of the animal (i.e., rate of movement of the animal), or pressure data from the animal (e.g., data from a capacitive pressure sensor), or biometric data of the animal (e.g., health data of the animal, such as heart rate, blood pressure, temperature, and the like).

In various embodiments, the remote network 705 may further comprise a social media platform 720 that provides interactive computer-mediated technologies that facilitate the creation or sharing of information, ideas, career interests and other forms of expression via virtual communities and networks, such as Facebook, Twitter, Instagram, and the like. Users may access social media services via web-based apps on devices, such cellphone, desktops, tablets, and laptops, or download services that offer social media functionality to their mobile devices (e.g., smartphones and tablets).

The social media platform 720 may be configured to receive the characteristic data from the host processor 710 and display the characteristic data. For example, the social media platform 720 may be capable of posting content containing the characteristic data, such as text, graphics, video and/or photos relating to the characteristic data. The social media platform 720 may also facilitate feedback from a user of the social media platform 720. For example, the social media platform 720 may provide an interface to request comments, a polling response, a thumbs up/down response, and the like, from the user as it relates to a particular post. In various embodiments, the social media platform 720 may share the feedback data with the host processor 705. The social media interface may comprise any number of interactive features, such as buttons, text boxes, and the like.

In various embodiments, the social media platform 720 may be used to initiate operation of the device 100. For example, a user of the social media platform 710 may use an interface within the social media platform 720 to generate a start signal. The social media platform 720 may transmit the start signal, via the host processor 710, to the device 100. In response to the start signal, the device 100 may generate a cue for the animal.

In various embodiments, the animal may wear a device equipped with a GPS (global positioning system) to provide the absolute location and/or time information of the animal. The location and/or time information may be transmitted to at least one of the access device 715, the host processor 710, the local device 100 or the social media platform 720. In various embodiments, the host processor 710 may provide a notification to an owner's access device 715 if the host processor 710 detects that the animal's location is outside of a specified range. For example, a notification may be sent to the owner's device to indicate that the animal's location is outside the owner's home or the owner's yard.

In various embodiments, the system 100 may further comprise a sub-processor 800 to execute a machine learning algorithm that has the ability to automatically learn and improve from experience without being explicitly programmed. The sub-processor 800 may be realized by the microprocessor 215 of the device 100, by the host processor 710, or a combination of the two. In the case where the sub-processor 800 is located within the device 100, the sub-processor 800 may be physically connected to the one or more sensors of the device 100.

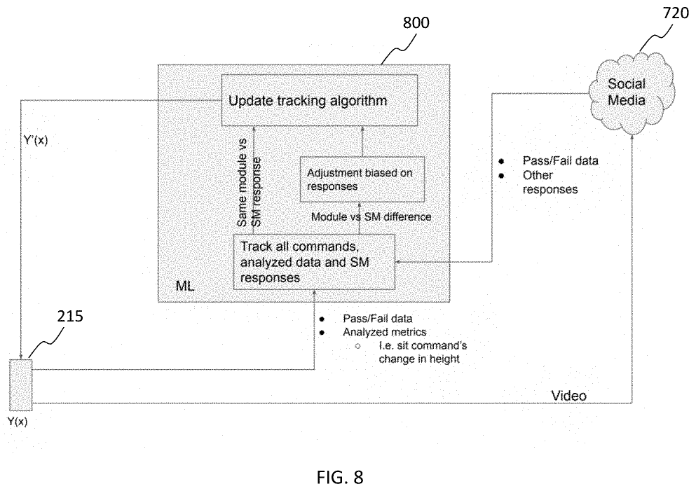

In an exemplary embodiment, and referring to FIG. 8, the machine learning algorithm may receive the characteristic data from the device 100, operational data (e.g., the start signal to provide a cue) and/or feedback data from the access device 715, and/or operational data (e.g., the start signal to provide a cue) and/or feedback data from the social media platform 720.

The machine learning algorithm may be programmed to generate learned data (i.e., refined data) according to the characteristic data and/or various feedback data streams. For example, the learned data may comprise data relating to at least one of a feature of the animal (i.e., breed, color, identity), a position (posture) of the animal (e.g., sitting, laying, standing), a movement of the animal (e.g., spinning jumping, etc.), or health of the animal. The health of the animal may correspond to information from the biometric sensors and/or information related to mood/emotion of the animal. Mood/emotion of the animal may be determined by analyzing the biometric data, observing the animal (e.g., via video or photos), or a combination of the two.

The machine learning algorithm may be realized by employing any suitable type of machine learning. For example, in one embodiment, the machine learning algorithm may comprise a supervised machine learning algorithm to apply what has been learned in the past to new data using labeled examples to predict future events. Starting from the analysis of a known training dataset, the learning algorithm produces an inferred function to make predictions about the output values. The system is able to provide targets for any new input after sufficient training. The learning algorithm can also compare its output with the correct, intended output and find errors in order to modify the model accordingly.

In another embodiment, the machine learning algorithm may comprise an unsupervised machine learning algorithm, which may be used when the information used to train is neither classified nor labeled. Unsupervised learning studies how systems can infer a function to describe a hidden structure from unlabeled data. The system doesn't figure out the right output, but it explores the data and can draw inferences from datasets to describe hidden structures from unlabeled data.

In another embodiment, the machine learning algorithm may comprise a semi-supervised machine learning algorithm fall that uses both labeled and unlabeled data for training—typically a small amount of labeled data and a large amount of unlabeled data. The systems that use this method are able to considerably improve learning accuracy. Semi-supervised learning may be chosen when the acquired labeled data requires skilled and relevant resources in order to train it/learn from it. Otherwise, acquiring unlabeled data generally doesn't require additional resources.

In another embodiment, the machine learning algorithm may comprise a reinforcement machine learning algorithm that interacts with its environment by producing actions and discovers errors or rewards. Trial and error search and delayed reward are the most relevant characteristics of reinforcement learning. This method allows machines and software agents to automatically determine the ideal behavior within a specific context in order to maximize its performance. Simple reward feedback is required for the agent to learn which action is best; this is known as the reinforcement signal.

In one operation, and referring to FIG. 8, assuming that the device 100 issues a “sit” command, device 100 may initially utilize the microcontroller 215 in conjunction with data from the sensors to track the animal and determine a pass or fail based on a machine learning algorithm Y(x). Video of the animal's response to the command may also be uploaded to the social media platform 720 where crowd sourced data (feedback data) will be collected and the characteristic data and the feedback data will be sent to the sub-processor 800 and analyzed by the machine learning algorithm. The original device data (characteristic data) and feedback data may then be compared against each other and, depending on the discrepancies, the sub-processor 800 may refine the machine learning algorithm Y(x) and generate an updated algorithm Y′(x) accordingly. The updated algorithm Y′(x) may be sent to the device 100 to replace Y(x) to be used for the next issued command.

In various embodiments, information from the device 100 may be communicated to the social media platform 720, a website or application that allows people to communicate and exchange ideas and information. The information can then be observed by an entity that is able to access the social media platform 720. In some embodiments, the entity may provide feedback through the social media platform 720, which may be communicated to the host processor 710 to be stored and/or used for further analysis. For example, a video stream may be transmitted to the social media platform 720 and the entity will be asked if the video represented a particular motion. The entity's response is entered and communicated to the host processor 710, which receives the response and uses the data to refine the machine learning algorithm.

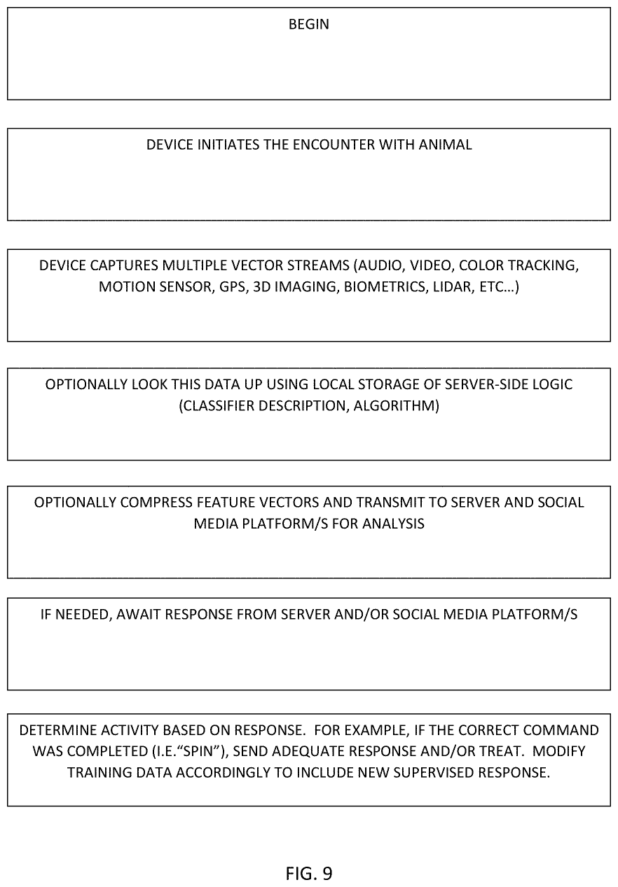

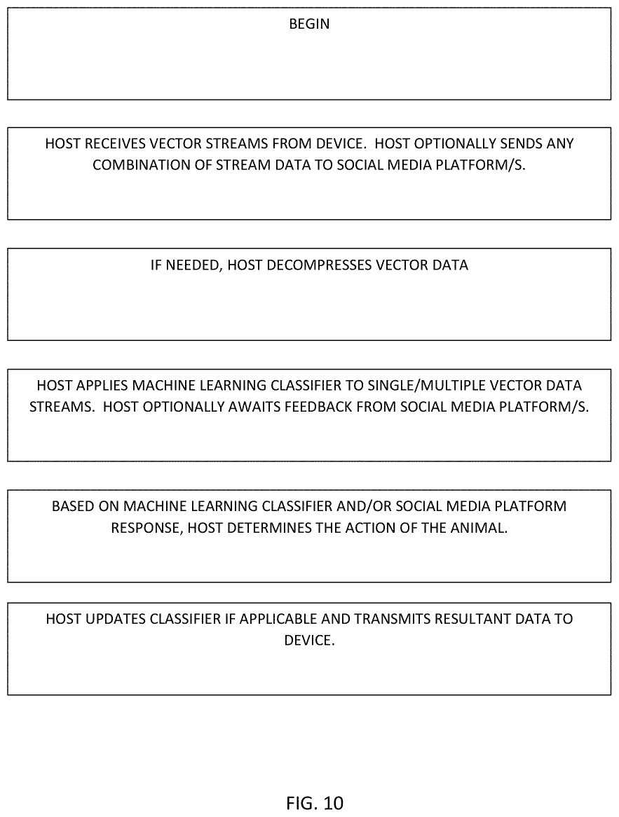

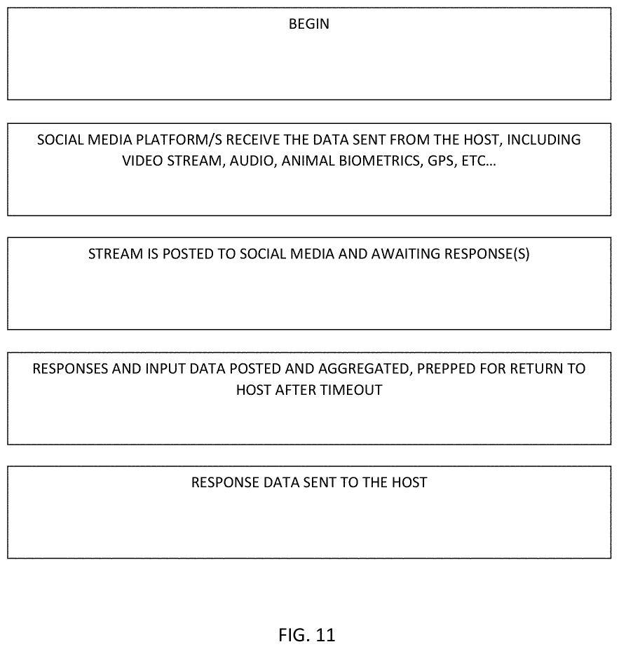

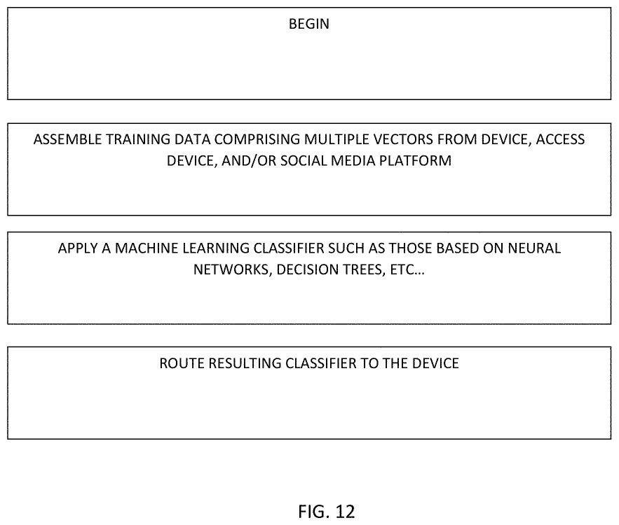

In various operations, and referring to FIGS. 9-13, the system 100 may use the device 100 capture characteristic data of the animal (FIG. 9), transmit the characteristic data to the host platform 710 (FIG. 10), transmit the characteristic data to the social media platform 720 (FIG. 11), and analyze the characteristic data and refine the machine learning algorithm using the feedback data from the access device and/or the social media platform 720 (FIG. 12).

For example, the device 100 may generate a “sit” cue/command for the animal and record the animal's response in the form of a video (i.e., characteristic data). The device 100 may then transmit the video to the host processor 710, wherein the host processor 710 transmits the video to the social media platform 720 and the social media platform 720 “posts” the video and facilitates users to provide feedback on whether or not the animal performed the “sit” command. For instance, users may use the “thumbs up” button to indicate a correct response, in that the animal performed a sit command in the video, or use the “thumbs down” button to indicate an incorrect response. After a period of time, the social media platform 720 may provide the feedback data (e.g., the number of “thumbs up” and the number of “thumbs down”) to the machine learning algorithm. The machine learning algorithm may utilize the feedback data and the original characteristic data to refine its definition of a “sit” command and provide the learned/refined data to the device 100. In various embodiments, the device 100 may incorporate the learned data into the microcontroller 215.

In various embodiments, the device 100 may be configured to analyze some combination of characteristic data of the animal in order to determine the optimum time frame for interaction. For example, the device 100 may determine that heart rate and blood pressure sensor data indicate that the animal may benefit from interaction, and will permit any remote or autonomous start signal to initiate the cue. Alternately, the device 100 may determine that some combination of characteristic data indicates that the animal may not benefit from interaction. For example, the device 100 may determine that heart rate, blood pressure, and acceleration data indicate that the animal is sleeping, and will prevent any remote or autonomous start signal from initiating the cue.

In various embodiments, the device 100 may combine stored data from the host processor 710 with characteristic data of the animal in order to determine the optimum time frame for interaction. For example, the device 100 may determine that stored data from the host processor indicates that early morning interactions with the animal have been the most successful, and combine this information with characteristic data to confirm that the cue may be initiated. Alternately, the device 100 may be configured to extend interactions into time frames that have historically proven to be least successful. For example, the device 100 may determine that stored data from the host processor 710 indicates that early afternoon interactions with the animal have been the least successful, and combine this information with characteristic data to increase the frequency of interactions during this time frame to attempt to improve the animal's response.

In various embodiments, the device 100 may be configured to analyze any combination of characteristic data and stored data to aid in determining the ongoing health of the animal. For example, the device 100 may continuously monitor the heart rate of the animal, and compare the heart rate with stored data on the host processor 710 to monitor for sickness, dehydration, poor diet, or the like. Alternately, the device 100 may monitor the activity level and posture of the animal and compare with stored data on the host processor 710 to monitor for physical ailments, such as arthritis, injury, or the like.

In various embodiments, characteristic and stored data related to the ongoing health of the animal may be communicated to a social media platform 720, and the social media platform 720 “posts” the data and facilitates users to provide feedback on whether or not a potential condition can be recognized. For instance, users may use the “possible joint inflammation” button to indicate a condition that can be recognized by a combination of video stream and historical data visualization. The social media platform 720 may provide the feedback data (e.g., the number of “possible joint inflammation” and the number of “animal appears healthy”) to the machine learning algorithm. The machine learning algorithm may utilize the feedback data, characteristic data, and historical data to refine its definition of a potential ongoing health condition and provide the learned/refined data to the device 100. In various embodiments, the device 100 may incorporate the learned data into the microcontroller 215. In some embodiments, the device 100 and/or host processor 705 may transmit notification to one or multiple access devices 715 of a potential health condition.

In various embodiments, the device 100 may be configured to compare characteristic data from before and after interactions to aid in determining the “success” of the interaction. For example, the device 100 may receive characteristic data prior to an interaction that indicates a particular heart rate, blood pressure, and activity level that may be associated with a degree of restlessness or anxiety. The device 100 may compare this data with the characteristic data following the interaction. Changes generally associated with a reduced degree of restlessness or anxiety (e.g., lower heart rate and/or blood pressure) may be associated with some degree of “success”. Alternately, changes generally associated with an increased degree of restlessness or anxiety (e.g., increased heart rate and/or blood pressure) may be associated with some degree of “failure”.

In various embodiments, characteristic data may used to aid in determining the “mood” of the animal. For example, the device 100 may generate a command and transmit the animal's response in the form of a video. The device 100 may then transmit the video to the host processor 710, wherein the host processor 710 transmits the video to the social media platform 720 and the social media platform 720 “posts” the video and facilitates users to provide feedback on what kind of mood the animal appeared to be in. For instance, user may use the “happy” button to indicate a more positive response, or use a “sad” or button to indicate a less positive, or negative, response. The social media platform 720 may provide the feedback data (e.g., the number of “happy” or “sad”) to the machine learning algorithm. The machine learning algorithm may utilize the feedback data and the original characteristic data to refine its definition of “happy” or “sad” and provide learned/refined data to the device 100. In various embodiments, the device 100 may incorporate the learned data into the microcontroller 215.

In various embodiments, the social media platform 720 may initiate activation of the device 100. For example, the social media platform 720 may involve active third parties with access to the host processor 710 in which the parties interact with the remote network 705 to transmit a signal to the device 100. Active third parties may have access to content initially published by the social media platform 720 via the host processor 710. Any interaction by a third party participant with the host processor 710 using the social media platform 720 is processed by host processor 710. Some examples may include direct messages, posted comments, reactions to published media, reactions to live media. In the event that these interactions occur, the host processor 710 may transmit a start signal to the device 100.

For example, given a third party participant active with the social media platform 720 reacts with a positive vote on a video published by the social media platform 720, then the host processor 710 detects the positive vote and may transmit a start signal to device 100, wherein the start signal initiates a cue/command for the animal.

In one operation where the machine learning algorithm is local to the device 100, the device 100 is initiated, and a training sequence begins. The data stream (h) is transmitted to the machine learning component (g). Upon determining the result of the training sequence, the device 100 initiates the appropriate output (u), where the output may provide a treat to the animal.

In one operation where the machine learning algorithm is shared between the device 100 and the remote network 705, the device 100 is initiated, and a training sequence begins. The data stream (h) is transmitted to the machine learning component (g) and to the handheld device (y) connected to the cloud data server/platform. The machine learning component (g) incorporates the response data input from the handheld device (y). Upon determining the result of the training sequence, the device initiates the appropriate output (u). For example, following training sequence initiation, the data is transmitted to the machine learning component and the cloud server/platform. The machine learning component (g) receives the response data input from the cloud server/platform, and incorporates this data into the machine learning algorithm. Upon determining the result of the training sequence, the device initiates the appropriate output (u) where the output may provide a treat to the animal.

In one operation where the access device 715 bypasses the machine learning algorithm, the device 100 is initiated, and a training sequence begins. The data stream (h) is transmitted to the handheld device (y) connected to the cloud data server/platform. Based on the response data input from the handheld device (y), the device initiates the corresponding output (u). For example, upon training sequence initiation, the data stream is transmitted to the handheld device (y). A user or any combination of users view the sequence and corresponding data, and generate the response data (i.e., ‘success’, ‘failure’, ‘sit’, spin'). The response data is returned to the device (x) through the cloud server/platform, and the device 100 processes the response data and subsequently initiates the appropriate output (u).

In one operation where the social media platform 720 bypasses the machine learning algorithm, the device (x) is initiated, and a training sequence begins. The data stream is transmitted to the cloud data server/platform, and is subsequently passed into any combination of Social Media Platforms (s) via API. Upon receiving response data input from the Social Media Platforms(s), the appropriate output is computed in the cloud server/platform. The output is transmitted to the device (x), and the appropriate output (u) is initiated, where the output may provide a treat to the animal.

In one operation where the machine learning algorithm uses data from the device 100, the access device 715, and the social media platform 720, the device 100 is initiated, and a training sequence begins. The data stream (h) is transmitted to the machine learning component (g) and to the cloud data server/platform. The cloud data server/platform then routes the data stream to Social Media (s) via API, and to the handheld device (y) via the platform app. The machine learning component (g) incorporates the response data input from the handheld device (y) and the Social Media platforms (s). Upon determining the result of the training sequence, the device initiates the appropriate output (u). For example, following training sequence initiation, the data is transmitted to the machine learning component and the cloud server/platform. The machine learning component (g) receives the response data input from the cloud server/platform, and incorporates this data into the machine learning algorithm. Upon determining the result of the training sequence, the device 100 initiates the appropriate output (u), where the output may provide a treat to the animal.

In the foregoing description, the technology has been described with reference to specific exemplary embodiments. The particular implementations shown and described are illustrative of the technology and its best mode and are not intended to otherwise limit the scope of the present technology in any way. Indeed, for the sake of brevity, conventional manufacturing, connection, preparation, and other functional aspects of the method and system may not be described in detail. Furthermore, the connecting lines shown in the various figures are intended to represent exemplary functional relationships and/or steps between the various elements. Many alternative or additional functional relationships or physical connections may be present in a practical system.

The technology has been described with reference to specific exemplary embodiments. Various modifications and changes, however, may be made without departing from the scope of the present technology. The description and figures are to be regarded in an illustrative manner, rather than a restrictive one and all such modifications are intended to be included within the scope of the present technology. Accordingly, the scope of the technology should be determined by the generic embodiments described and their legal equivalents rather than by merely the specific examples described above. For example, the steps recited in any method or process embodiment may be executed in any order, unless otherwise expressly specified, and are not limited to the explicit order presented in the specific examples. Additionally, the components and/or elements recited in any apparatus embodiment may be assembled or otherwise operationally configured in a variety of permutations to produce substantially the same result as the present technology and are accordingly not limited to the specific configuration recited in the specific examples.

Benefits, other advantages and solutions to problems have been described above with regard to particular embodiments. Any benefit, advantage, solution to problems or any element that may cause any particular benefit, advantage or solution to occur or to become more pronounced, however, is not to be construed as a critical, required or essential feature or component.

The terms “comprises”, “comprising”, or any variation thereof, are intended to reference a non-exclusive inclusion, such that a process, method, article, composition or apparatus that comprises a list of elements does not include only those elements recited, but may also include other elements not expressly listed or inherent to such process, method, article, composition or apparatus. Other combinations and/or modifications of the above-described structures, arrangements, applications, proportions, elements, materials or components used in the practice of the present technology, in addition to those not specifically recited, may be varied or otherwise particularly adapted to specific environments, manufacturing specifications, design parameters or other operating requirements without departing from the general principles of the same.

The present technology has been described above with reference to an exemplary embodiment. However, changes and modifications may be made to the exemplary embodiment without departing from the scope of the present technology. These and other changes or modifications are intended to be included within the scope of the present technology, as expressed in the following claims.

As the claims hereinafter reflect, inventive aspects may lie in less than all features of a single foregoing disclosed embodiment. Thus, the hereinafter expressed claims are hereby expressly incorporated into this Detailed Description of the Drawings, with each claim standing on its own as a separate embodiment of the present technology. Furthermore, while some embodiments described herein include some but not other features included in other embodiments, combinations of features of different embodiments are meant to be within the scope of the present technology, and form different embodiments, as would be understood by those skilled in the art.

1. A system for interacting with an animal, comprising:
a local device configured to provide a cue directed to the animal and generate characteristic data of the animal;
a remote network in communication with the local device and configured to:
receive the characteristic data and generate first feedback data; and
execute a machine learning algorithm to generate learned data based on the first feedback data and the characteristic data;
wherein the local device is further configured to operate according to the learned data.

2. The system of claim 1, wherein the characteristic data comprises at least one of image data of the animal, audio data of the animal, orientation data of the animal, position data of the animal, acceleration data of the animal, pressure data from the animal, or biometric data of the animal.

3. The system of claim 1, wherein the remote network comprises a host platform and a social media platform, wherein the host platform receives the characteristic data from the local device, delivers the characteristic data to the social media platform, and delivers the learned data to the local device.

4. The system of claim 3, wherein the social media platform is configured to share the characteristic data with the host platform and facilitate an interaction to generate the first feedback data.

5. The system of claim 3, wherein the local device initiates the cue in response to a start signal from the social media platform.

6. The system of claim 3, further comprising an access device configured to communicate with the host platform, view the characteristic data, and facilitate receipt of second feedback data from a user of the access device.

7. The system of claim 6, wherein the local device initiates the cue in response to a start signal from the access device.

8. The system of claim 1, wherein the local device initiates the cue autonomously.

9. The system of claim 1, wherein the learned data comprises data relating to at least one of:
a feature of the animal, a position of the animal, a movement of the animal, or health of the animal.

10. A system for interacting with an animal, comprising:
a local device configured to provide a cue directed to the animal and generate characteristic data of the animal;
a host platform in communication with the local device and configured to receive the characteristic data;
a social media platform in communication with the host platform and configured to facilitate an interaction to generate first feedback data and transmit the first feedback data to the host platform; and
a processor configured to execute a machine learning algorithm to generate learned data based on the first feedback data and the characteristic data;
wherein the local device is further configured to operate according to the learned data.

11. The system of claim 10, further comprising an access device configured to communicate with the host platform, view the characteristic data, and facilitate receipt of second feedback data from a user of the access device.

12. The system of claim 11, wherein the local device initiates the cue in response to a start signal from the access device.

13. The system of claim 10, wherein the characteristic data comprises at least one of image data of the animal, audio data of the animal, orientation data of the animal, position data of the animal, acceleration data of the animal, pressure data from the animal, or biometric data of the animal.

14. The system of claim 10, wherein the learned data comprises data relating to at least one of: a feature of the animal, a position of the animal, a movement of the animal, or health of the animal.

15. The system of claim 10, wherein the local device initiates the cue autonomously.

16. The system of claim 10, wherein the local device comprises a plurality of sensors to generate the characteristic data and the processor is physically connected to the plurality of sensors.

17. A system for interacting with an animal, comprising:
a local device configured to provide a cue directed to the animal and generate characteristic data of the animal;
a host platform in communication with the local device and configured to receive the characteristic data; and
a social media platform in communication with the host platform and configured to:
display the characteristic data;
facilitate an interaction to generate feedback data based on the characteristic data; and
transmit the feedback data to the host platform; and
wherein the local device is further configured to operate according to the feedback data.

18. The system of claim 17, wherein:
the characteristic data comprises at least one of image data of the animal, audio data of the animal, orientation data of the animal, position data of the animal, acceleration data of the animal, pressure data from the animal, or biometric data of the animal; and
the feedback data comprises data relating to at least one of: a feature of the animal, a position of the animal, a movement of the animal, or health of the animal.

19. The system of claim 17, wherein the local device initiates the cue in response to a start signal from the social media platform.

20. The system of claim 17, further comprising a processor in communication with the local device and the social media platform, wherein the processor is configured to execute a machine learning algorithm to generate learned data based on the feedback data and the characteristic data, and wherein the local device is further configured to operate according to the learned data.
